(19) United States
(12) Patent Application Publication
(10) Pub. No.: US 2009/0029403 A1
(43) Pub. Date: Jan. 29, 2009

(54) Sesnsor for detecting a toxic or hazardous gas mixture and operating method

(76) Inventors: Maximilian Fleischer, Hoehenkirchen (DE); Mirko Lehmann, Ebmat-Kappel (CH); Elfriede Simon, Munich (DE)

Correspondence Address: STAAS & HALSEY LLP, SUITE 700, 1201 NEW YORK AVENUE, N.W., WASHINGTON, DC, 20005, US

(21) Appl. No.: 12/078,794

(22) Filed: Apr. 4, 2008

(30) Foreign Application Priority Data
Apr. 5, 2007 (DE) 102007016629.1

(51) Int. Cl. C12Q 1/02; C12M 1/34

(52) U.S. Cl. 435/29; 435/287.1

(57) ABSTRACT

A sensor for detecting a gas mixture which substantially comprises air and contains one or a plurality of gases that exhibit a disadvantageous effect on living organisms, comprising: a sensor chip composed of silicon for reading out at least one signal which is generated at a sensitive substance given the presence of one or a plurality of target gases in the measurement gas, a sensitive substance applied on the sensor chip, comprising living cells, which respond to target gas, a signal processing unit for evaluating the signals of the Si chip.

### CROSS REFERENCE TO RELATED APPLICATIONS

This application is based on and hereby claims priority to German Application No. 10 2007 016 629.1 filed on Apr. 5, 2007, the contents of which are hereby incorporated by reference.

### BACKGROUND OF THE INVENTION

The detection is intended to determine a toxic pollution or pollution that should be classified as hazardous in some other way, without the need to perform a differentiation of individual gases in a gas mixture. The gas atmosphere present in the environment is generally a gas mixture comprising a plurality of different gases. Corresponding gas sensors geared to a respective gas have hitherto been necessary in order to detect a composition of a gas mixture. Said gas sensors measure the presence of a target gas and possibly also the concentration thereof.

The previous approach of ascertaining and categorizing gases may perfectly well encounter its limits if a wide variety of variants of toxic or hazardous gases have to be detected and a measuring system therefore has to be of very complex configuration.

Precise knowledge of the target substance/s has hitherto been necessary for monitoring for hazardous substances. Gas sensors or a plurality of gas sensors can thus be used to detect these substances individually.

There have already been initial approaches with regard to using living organisms as an indicator of the presence of hazardous substances in air. A classic example is canaries, which have long been used in mining, have a high sensitivity to poisonous gases and can thereby protect human beings.

Approaches with regard to using living organisms and their biological receptors as sensors are known. Thus, by way of example, the antennae of Colorado beetles can be brought into electrical contact with the gate of field effect transistors. The reaction of a highly sensitive beetle antenna to gases or gas mixtures can be read out via the source-drain current of FETs.

FIGS. 2 and 3 respectively show a scheme of an experimental arrangement comprising a mount 10 for the antenna 9 of Colorado beetles 7. In this case, according to FIG. 2, the entire Colorado beetle is present during the detection, while a separate antenna 9 of the Colorado beetle 7 is used according to FIG. 3. The measurement is performed with the aid of an electrolyte 8. Measurement signals generated are read out via at least one FET which is correspondingly fixed to a mount.

FIG. 4 shows an arrangement according to FIG. 2 with the exemplary illustration of an electronic evaluation circuit having various amplifier, filter and converter units.

Approaches with regard to using cells per se as biological receptors are furthermore known in the medical sector, e.g. in oncology for improving the treatment of patients suffering from cancer diseases.

In the publication: Lehmann, M., Baumann, W., Brischwein, M., Gahle, H. J., Freund, I., Ehret, R., Drechsler, S., Palzer, H., Kleintges, M., Sieben, U., Wolf, B., 2001, “Simultaneous measurement of cellular respiration and acidification with a single CMOS ISFET, Biosensors & Bioelectronics”, 16, pages 195-203, essentially the pH value and the oxygen partial pressure are observed as parameters for the continuance of living tissue.

Furthermore, DE 19827957 C2 describes a method and a device for measuring a state variable.

DE 19920811A1 discloses a device for carrying out investigations on cell cultures.

Moreover, DE 10028692 A1 discloses a method for examining membrane-enclosed biocompartments.

The patent applications and publications presented here describe sensor chips and systems which are generally aimed either at the response of cells to known liquids, such as e.g. in the search for active substances in the development of medicaments or at the response of cells to unknown liquids, such as e.g. when monitoring water quality.

### SUMMARY

It is one possible object to provide a sensor and an operating method whereby it is possible to detect signals of living cells as receptors in the case of toxic or hazardous gases or gas mixtures present in a measurement gas.

The inventors developed insight that with this approach in contrast to previous approaches in gas sensor technology, objectives are achieved which do not necessitate selective detection of an individual gas, but rather enable examination of an environment with a gas mixture, with a statement about the harmfulness for living organisms. In this case, the detection is not constricted to one or a plurality of gases, rather an arbitrarily wide spectrum of possible gases are considered.

Applications are found e.g. in environmental measurement technology, where there is often the problem of measuring and being able to assess the pollution of a surrounding atmosphere. It is possible to ascertain whether the atmosphere contains e.g. pollution due to traffic, pollution due to industrial waste gases or else industrial exhaust air or generally contains constituents harmful to life, where the main emphasis here is placed on harmfulness to human beings. Other applications are found in defense against terrorism or workplace safety with the monitoring of harmful chemicals or mixtures, where gases or gas mixtures that correlate therewith are to be detected. This involves detecting whether substances harmful to life are in the environment, without the target substances having to be explicitly known.

Advantageous embodiments are in particular as follows:

The cells are situated directly on/in direct proximity to the chip region which reads out the cell signals.

The signals of the living cells are recorded by electrodes which forward them to chip regions at a distance from the cells, for signal detection purposes, i.e. read out the cell signals.

It is advantageous to use a fine-pored membrane having pore widths of typically 0.1-10 μm, which permits the admittance of gases to the sensor, but keeps away dust, aerosols and foreign bacteria and viruses. Said membrane can additionally be dimensioned in such a way that sufficient gas admittance to the cells is ensured, but a loss of moisture is reduced by the partial covering of the surface.

Devices which permit the cells to survive for relatively long times are situated in the housing. Said devices provide e.g. for supplying the cells with enough moisture so as to avoid drying out, and ensure a nutrient supply for the survival of the cells. If use is made of cells that require light to survive which are e.g. organisms based on photosynthesis, then it is also necessary to ensure a supply with light of the required spectral range.

The sensor chip is constructed in such a way that the cells on the chip are embedded into a gas-permeable matrix which brings about a better anchoring of the cells and fixes the cells on the sensor surface.

It is advantageous to equip the matrix for ensuring sufficient moisture.

A further advantageous configuration provides for the matrix to ensure the nutrient supply of the cells.

One configuration concerns the matrix, which contains a growth inhibitor (cytostatics) in order to prevent proliferation of the cells.

It is furthermore advantageous to add bactericides e.g. antibiotics, and fungicides, e.g. antimycotics to the matrix in order to prevent colonization by foreign bacteria and fungi and spores.

A particular configuration is that the matrix contains a sodium azide, streptomycin, penicillin, in order to prevent infestation by foreign bacteria.

A particular configuration is that the matrix contains the substances necessary for the survival of the cells.

It is particularly advantageous that the matrix contains substances such as e.g. proteins and enzymes, e.g. through addition of fetal serum, normal serum or glutamine.

Furthermore, a fine-pored membrane having pore widths of typically between 0.1 and 10 μm can be fitted directly on the matrix, which membrane permits the admittance of gases to the sensor but keeps away dust, aerosols and foreign bacteria and viruses.

Since the living cells can only survive in a liquid environment, the gas must undergo transition from the gaseous state to the liquid state in order to reach the cells. The solubility of gas with respect to liquid is determined by Henry's law. It should be taken into consideration here that diffusion in gases proceeds very much more rapidly than in liquids. The diffusion time of H+ protons for 1 mm in liquid is approximately 10 s. The larger the gas molecules, the longer this time becomes. For a fast response time of the sensor to e.g. poison gas, etc., therefore, the distance from the gas/liquid boundary to the cells on the sensor is to be made as short as possible. Furthermore, care must be taken to ensure that such access is present, since in the normal cell sensor systems such access is intended rather to be precluded in order not to contaminate the cells and the liquid.

It is advantageous that a distance between the living cells and the gas environment in the system under consideration, that is to say the distance from the gas/liquid boundary to the living cells, is less than 1 mm, preferably less than 500 μm.

One possibility of feeding measurement gas to the receptor or to the gas-sensitive unit, here the living cells, involves etching free the silicon chip at the rear side in small regions having a diameter from the range of 0.1 mm down to approximately 1-10 μm, such that the gas rapidly reaches the cell and the height of the liquid above the cells can nevertheless be chosen rather freely. If the holes are produced e.g. using traditional wet-chemical etching technology in the silicon, it should be taken into consideration that they have a V-shaped cross section. The indication of the diameter then means the narrowest location in the hole. The choice of the size and number of the “holes” would have to be made depending on the magnitude of the pressure of the liquid, which essentially results from the height and the flow rate and can be chosen such that the liquid does not emerge from the holes. Furthermore, it is expedient to configure the holes such that the cells do not pass through the holes; this can e.g. also be realized by a membrane on the Si surface. A further similar possibility provides for introducing passages into the walls of packages.

In this embodiment, too, in which the measurement gas passes rapidly to cells “from below”, without having to cover time-consuming diffusion distances through the liquid, it may be expedient to use one of the fine-pored membranes mentioned above in order to keep foreign particles away. Said membrane is then fitted over the pores.

As an alternative, use is made of a fluidic solution in which the interaction of gas and liquid does not take place directly over the sensor chip, but rather in a fluid channel before the sensor chip, which is made very thin (diameter of 10 μm to 1 mm). Said fluid channel is very long in order that a large amount of liquid comes into contact with the gas. The separating material of the fluid channel is then gas-permeable, of course, in order that the gas can pass into the liquid. This liquid provided with the gas is then brought to the cells and the gas is thus fed to the cells. For moving the liquid, a pump connected to the fluid channel can be used in order to lead the liquid “contaminated” with the gas over the sensor chip. As an alternative, the liquid can also be moved by other variants such as by a hydrostatic solution, by the force of gravity.

If a solution is used in which the cells are actively flushed with a fluid, it may be advantageous to interrupt the flushing of the cells in the meantime. During the time in which cells are flushed with the liquid exposed to the gas, the gas is transported to the cells. At the same time, however, metabolic products of the cells which are read out by the Si chip are also transported away and can therefore be measured only with a reduced sensitivity. If the flow is stopped, said metabolic products accumulate. It is then possible to detect more sensitively changes to the cells by the measurement of said metabolic products. On the other hand, this can also be used for stabilizing the measuring method if, rather than the absolute content of the metabolic products of the cells, now only the relative rise in the concentration thereof is measured. In this variant, the pick-ups in the silicon are no longer required to have a stable base line. It suffices for a change in the concentration of metabolic products to be measured by the pick-ups in the silicon.

Since each cell responds differently to the external gases, it is presumably expedient to use not just one sensor chip with an individual cell type, but rather a plurality thereof, the sensor chips differing by virtue of the other cells. If a device for monitoring locations is considered, for example, it is rather unlikely that there will be one cell which responds to all poison gases in a suitable manner. In other words one has a system having at least two sensor chips with different cell types, the signals of which, processed with one another, can trigger an alarm. As an alternative, for reasons of simplifying the construction, it is also possible to provide different cell lines on one chip, the aggregate signal of said cell lines then being carried out.

A cell line which is applied in a specific density on the read-out chip will have a specific harmful substance concentration at which it exhibits the strongest reactions. In order to extend the measurement range of a cell sensor, it will therefore be expedient to provide different individual sensors provided with different cell densities. Each individual sensor with a specific cell density is thus optimally sensitive for a specific concentration range of harmful substances.

The sensor system is equipped with an auto-calibration unit in order to set the zero point at regular time intervals on the monitoring sensors.

The contact of the cell-based sensor with hazardous and toxic gases is detected by recording the change in one or more of the parameters typical of cells, such as adhesion, detected by an impedance measurement, such as O₂ consumption, detected by an O₂ measurement, acidification, detected by a pH value measurement, electrical activity, detected by a potential change, or bioluminescence, detected by a measurement of the emitted light. These can then be compared with the signals of a second uncontaminated cell sensor.

Determining the time profile of the change in the parameters and evaluating the parameter kinetics.

Previous approaches for areas such as e.g. environmental measurement technology—air, water—or defense against terrorism, in relation to poison gas, are based on always using sensors for individual substances. If a large number of individual substances are at issue, a very large number of sensor elements have to be used which then make the construction expensive and susceptible to disturbance.

In some applications, the possible poisonous/disturbing materials of corresponding substances are not known or too diverse, such that the approach via detection of individual substances is not practicable.

The approach described achieves both together. By using living cells the cross-sectional receptor goes into action which measures a multiplicity of substances or the effect thereof on living cells with one sensor element.

At the same time there is the possibility, through a suitable choice of the cell line, to adapt the relative distribution of the sensitivities to the different harmful substances to human sensitivity.

Cells require a certain temperature range to be maintained in order to be able to live. In order to be able to implement the use of the proposed construction in diverse environments, therefore, the use of heating or cooling arrangements for maintaining a constant predetermined temperature of the construction is also optionally provided.

Cells often have a limited lifetime. If the latter is too short for the application, a plurality of chips of identical type can be provided. In this case, by way of example, one chip is used for the measurement, while the other chips are not used. In this case, the non-use of the chips is ensured for example by the chips not being exposed to the measurement gas, or by the chips being deactivated by the use of a growth inhibitor for the cells or the choice of local temperature. In the case where the exhaustion of the measuring capability of the chip is indicated or after a fixedly predetermined time, the next chip is then used. For this purpose, the deactivating influences are removed and the signal of the chip activated in this way is read out.

An evaluation unit is connected to the chips. Said evaluation unit will perform an evaluation of the signals of the individual chips.

If only an individual chip is read, no statement can be made about the type of gas/gas group implementing the cells since it is not possible to identify whether there is a low concentration of a gas having a great influence on the cells or a higher concentration of a gas having a less pronounced influence on the cells. If the two gases having an equally great influence on the cells are present, no statement can be made about which gas is involved. If a plurality of different chips whose cells react differently to different gases are then present and operated, different signals are obtained. By an evaluation of the different signals in the evaluation unit, e.g. by a rule-based evaluation or using pattern recognition methods, given knowledge of the sensitivities of the individual chips, it is then possible to effect a classification of the events with regard to a statement about the type of gases/gas groups present.

### BRIEF DESCRIPTION OF THE DRAWINGS

These and other objects and advantages of the present invention will become more apparent and more readily appreciated from the following description of the preferred embodiments, taken in conjunction with the accompanying drawings of which:

FIG. 1 shows an arrangement according to the invention, a sensor being illustrated in section,

FIGS. 2, 3 and 4 show arrangements that reproduce the related art.

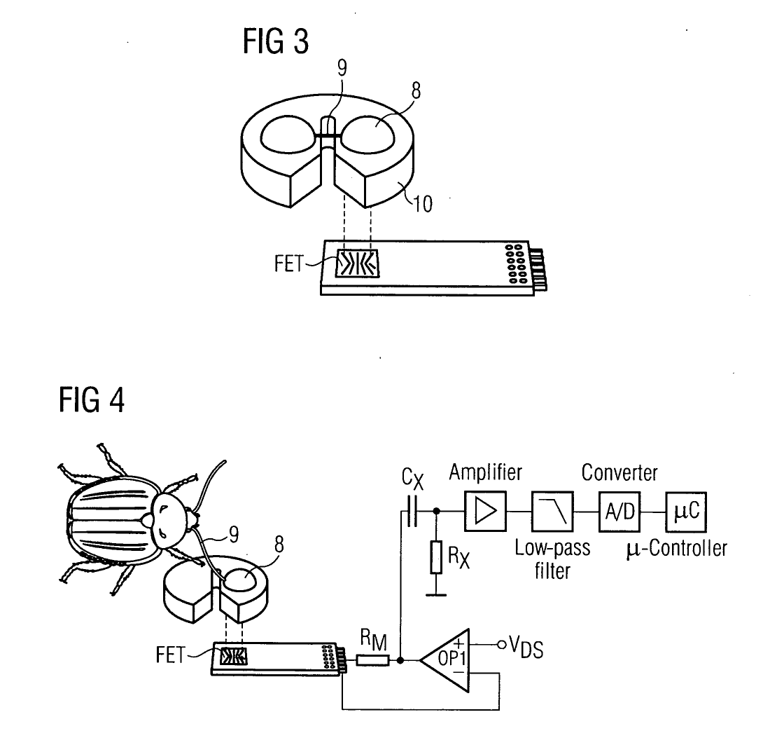

### DETAILED DESCRIPTION OF THE PREFERRED EMBODIMENT

Reference will now be made in detail to the preferred embodiments of the present invention, examples of which are illustrated in the accompanying drawings, wherein like reference numerals refer to like elements throughout.

The sensor system is operated together with an auto-calibration unit in order to set the zero point at regular time intervals on the monitoring sensors. The sensor comprises at least three separate but structurally identical sensor units exposed to ambient air/measurement gas and three separate but structurally identical units brought into contact with uncontaminated air. A unit that fails on account of a disturbance, such as e.g. contamination with foreign cells, can then be identified for plausibility reasons (two identical and one different reaction pattern).

The sensor system is equipped with new cell sensors at regular intervals, such as typically every 5-40 days.

The sensor system is constructed in such a way that the sensor chip can be replaced in a simple manner with the aid of a laterally fitted plug system.

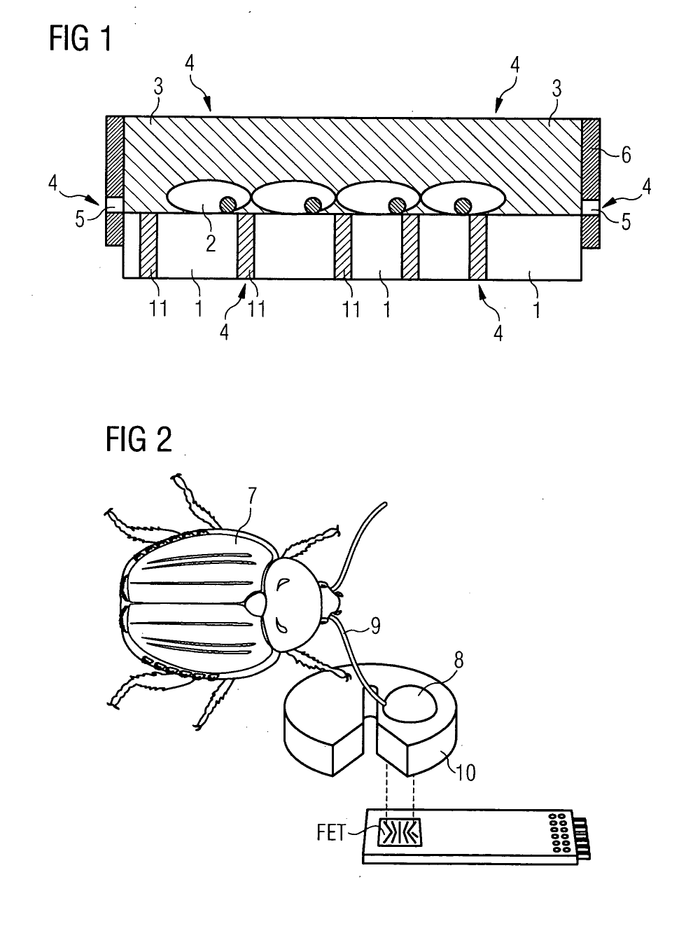

FIG. 1 shows a sensor in one possible cross-sectional form. Arranged centrally there are symbolically four oversized living cells of oval shape having a circular respective cell nucleus likewise illustrated in enlarged fashion.

The living cells 2 bear on the upper side of the silicon chip 1, in which case it should be taken into consideration that the cells have to be embedded in liquid or in a moist medium for relatively long survival.

The living cells 2 are embedded in a matrix which advantageously contains a nutrient medium 3. A device for stabilizing the cells and the nutrient medium 3 is represented here by the frame 6.

A required access of measurement gas 4 to the sensitive layer, the receptor, here the living cells 2, is ensured through the openings or the passages 11. The latter can permit an inflow of measurement gas 4 from below to the living cells 2 through the Si chip 1. In addition, passages 11 can be positioned laterally in the frame 6. The minimized diffusion distance through a liquid substance surrounding the living cells or a correspondingly thin layer of said substances is essential.

The invention has been described in detail with particular reference to preferred embodiments thereof and examples, but it will be understood that variations and modifications can be effected within the spirit and scope of the invention covered by the claims which may include the phrase “at least one of A, B and C” as an alternative expression that means one or more of A, B and C may be used, contrary to the holding in Superguide v. DIRECTV, 69 USPQ2d 1865 (Fed. Cir. 2004).

1. A sensor for detecting a gas mixture which comprises air and a target gas that exhibits an effect on living organisms, comprising:
a sensitive substance comprising living cells, which respond to the target gas, the sensitive substance generating a signal in the presence of the target gas;
a silicon sensor chip to read the signal which is generated by the sensitive substance, the sensitive substance being applied to the sensor chip; and
a signal processing unit to evaluate the signal read by the sensor chip.

2. The sensor as claimed in claim 1, further comprising a device to supply the living cells with nutrients and moisture in order to optimize a lifetime of the cells.

3. The sensor as claimed in claim 1, further comprising a device to supply the living cells with light in order to optimize a lifetime of the cells.

4. The sensor as claimed in claim 1, wherein the living cells on the sensor chip are embedded in a gas-permeable matrix which fixes the cells to the sensor chip.

5. The sensor as claimed in claim 1, further comprising a device to supply the living cells with a cryostatic growth inhibitor to prevent proliferation of the cells.

6. The sensor as claimed in claim 1, further comprising a device to supply the living cells with a bactericide and/or a fungicide to prevent colonization by foreign bacteria, fungi or spores.

7. The sensor as claimed in claim 1, further comprising a device to supply the living cells with at least one of sodium azide, streptomycin, and penicillin to prevent infestation by foreign bacteria.

8. The sensor as claimed in claim 1, further comprising a device to supply the living cells with fetal serum, normal serum or glutamine to provide the living cells with proteins and enzymes.

9. The sensor as claimed in claim 1, further comprising a device to maintain a constant predetermined temperature at the sensor chip.

10. The sensor as claimed in claim 1, further comprising a fine-pored membrane having a pore width of approximately 0.1 μm-10 μm, which permits the admittance of the gas mixture to the sensor, but blocks harmful substances selected from the group consisting of dust, aerosols, foreign bacteria and viruses.

11. The sensor as claimed in claim 10, wherein the fine-pored membrane comprises at least one polymer selected from the group consisting of polyfluorinated hydrocarbons, polypropylenes, polyethylenes, polyamides and polyimides.

12. The sensor as claimed in claim 1, wherein the living cells are situated directly on the sensor chip, such that the signal can be read out.

13. The sensor as claimed in claim 1, further comprising electrodes to transmit signals from the living cells and to a read-out chip region.

14. The sensor as claimed in claim 1, wherein the distance between the living cells and the gas mixture is less than 1 mm.

15. The sensor as claimed in claim 14, wherein the distance between living cells and the gas mixture is less than 500 μm.

16. The sensor as claimed in claim 1, wherein at least one passage is provided through the sensor chip or at boundaries of the sensitive substance to allow the gas mixture to reach the sensitive substance.

17. The sensor as claimed in claim 16, wherein the at least one passage has a diameter of approximately 5-10 μm.

18. The sensor as claimed in claim 1, wherein
the gas mixture is dissolved in a liquid, and
at least one narrow elongated fluid channel having a diameter within the range of 10 μm to 1 mm actively transports the liquid to the living cells.

19. The sensor as claimed in claim 18, wherein an externally generated flow of the liquid is passed over the sensor chip.

20. The sensor as claimed in claim 1, wherein
a plurality of sensor chips are combined, and
the sensor chips each contain different types of living cells.

21. The sensor as claimed in claim 1, wherein a plurality of sensor chips are present, on which there are different densities of living cells.

22. The sensor as claimed in claim 1, wherein different cell lines are situated on the sensor chip.

23. A method for detecting a gas mixture which comprises air and a target gas that exhibits an effect on living organisms, comprising:
exposing a sensor to the gas mixture, the sensor comprising:
a sensitive substance comprising living cells, which respond to the target gas, the sensitive substance generating a signal in the presence of the target gas;
a silicon sensor chip to read the signal which is generated by the sensitive substance, the sensitive substance being applied to the sensor chip; and
a signal processing unit to evaluate the signal read by the sensor chip; and
setting a zero point for the sensor at regular time intervals using an auto-calibration unit.

24. The method as claimed in claim 23, wherein
an active sensor producing an active signal is exposed to the gas mixture and a reference sensor producing a reference signal is unburdened by the gas mixture, and
the active and reference signals are compared.

25. The method according to claim 23, further comprising:
flushing of the cells with a fluid in which the gas mixture is dissolved; and
periodically interrupting a supply of the fluid.

26. The method as claimed in claim 25, wherein metabolic products of the cells are measured during interruption of the fluid.

27. The method as claimed in claim 23, wherein
there are a plurality of sensors, and
the sensors are operated sequentially such that when a measuring capability of an operated sensor is exhausted, a new chip is activated.

28. The method as claimed in claim 27, wherein the new chip is activated by removing a growth inhibitor.

29. The method as claimed in claim 23, wherein
a plurality of different sensors producing respective signals are exposed to the gas mixture, and
the evaluation unit compares the signals from the different sensors and obtains therefrom more extensive information about the gases present in the gas mixture.

30. A sensor for detecting a gas mixture which comprises air and a target gas that exhibits an effect on living organisms, comprising:
a sensitive substance comprising living cells, which respond to the target gas, the sensitive substance generating a signal in the presence of the target gas;
a semiconductor sensor chip to read the signal which is generated by the sensitive substance, the sensitive substance being applied to the sensor chip; and
a signal processing unit to evaluate the signal read by the sensor chip.
